(19) United States
(12) Patent Application Publication
(10) Pub. No.: US 2004/0127430 A1
(43) Pub. Date: Jul. 1, 2004

(54) Chiral monophosphorus compounds and their transition metal complexes

(76) Inventors: Benjamin Meseguer, Tarragona (ES); Hans-Christian Militzer, Odenthal (DE); Sergio Castillon, Tarragona (ES); Carmen Claver, Tarragona (ES); Ester Guiu, Tarragona (ES)

Correspondence Address: BAYER CHEMICALS CORPORATION, PATENT DEPARTMENT, 100 BAYER ROAD, PITTSBURGH, PA, 15205-9741, US

(21) Appl. No.: 10/660,150

(22) Filed: Sep. 11, 2003

(30) Foreign Application Priority Data
Sep. 12, 2002 (DE) 10242351.2

(51) Int. Cl. A61K 31/70; A61K 31/695; A61K 31/665

(52) U.S. Cl. 514/23; 514/63; 514/99; 536/109; 549/214; 549/216

(57) ABSTRACT

The present invention relates to chiral monophosphorus compounds and their transition metal complexes, to a process for preparing chiral monophosphorus compounds and their transition metal complexes and also to their use in asymmetric syntheses.

### BACKGROUND OF THE INVENTION

1. Field of the Invention

The present invention relates to chiral monophosphorus compounds and their transition metal complexes, to a process for preparing chiral monophosphorus compounds and their transition metal complexes and also to their use in asymmetric syntheses.

2. Brief Description of the Prior Art

Enantiomerically enriched chiral compounds are valuable starting substances for preparing agrochemicals and pharmaceuticals. Asymmetric catalysis has gained great industrial significance for the synthesis of such enantiomerically enriched chiral compounds.

Recent publications in the field of asymmetric synthesis show clearly that transition metal complexes of monophosphorus compounds are very suitable as catalysts in reactions conducted asymmetrically, in particular in asymmetric hydrogenations of C═O, C═N and C═C bonds. For example, A. Alexakis, Tetrahedron Asymmetry, 1997, 8, 3193-3196; W. Chen, J. Xiao, Tetrahedron Letters, 42, 2001, 2897-2899; M. Reetz, G. Mehler, Angew. Chem., 2000, 112, 4047-4049 and WO-A 01/94278 disclose the use of optically active monophosphites or their transition metal complexes for asymmetric hydrogenations.

The use of optically active monophosphoramidites or their transition metal complexes in asymmetric syntheses is disclosed, for example, by M. van den Berg et al., J. Am. Chem. Soc., 2000, 122, 11539-11540, WO-A 02/04466; H. Waldmann, Chem. Eur. J. 2000, 6, 671-675; the use of chiral monophosphonites, for example, by C. Claver et al., Chem. Commun., 2000, 961-962.

A disadvantage of the cited monophosphorus compounds is that steric and electronic variation of the ligand framework which is necessary for optimization and adaptation of the ligands and therefore the catalyst for a given substrate is possible only to a very limited extent, and only by numerous, complicated synthetic steps. This disadvantage distinctly limits industrial utilization of such ligands and the catalysts preparable therefrom.

There was therefore the need to develop a ligand system whose steric and electronic properties can be easily varied and which is based on monophosphorus compounds, and whose transition metal complexes, as catalysts in asymmetric synthesis, in particular asymmetric hydrogenations, enable high enantioselectivities.

### SUMMARY OF THE INVENTION

Compounds have now been found of the formula (I)

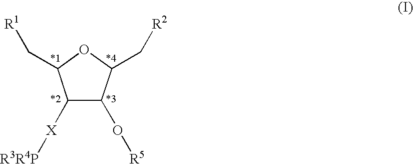

where

*1, *2, *3 and *4 are each independently a stereogenic carbon atom which has R- or S-configuration,

X is absent or is oxygen and

R¹ and R² may each independently be hydrogen, C₁-C₂₀-alkyl, C₁-C₂₀-fluoroalkyl, C₂-C₂₀-alkenyl, C₄-C₂₄-aryl, C₅-C₂₅-arylalkyl, C₆-C₂₆-arylalkenyl or NR⁶R⁷, OR⁷, —(C₁-C₈-alkyl)-OR⁷, —(C₁-C₈-alkyl)-NR⁶R⁷ or —O₂CR⁷,

where R⁶ and R⁷ are each independently C₁-C₈-alkyl, C₅-C₁₅-arylalkyl or C₄-C₁₄-aryl, or R⁶ and R⁷ together are a cyclic amino radical having a total of 4 to 20 carbon atoms,

or R¹ and R² are each independently radicals of the formula (IIa)

—R⁸—SiR⁹R¹⁰R¹¹ (IIa)

where

R⁸ is absent or is oxygen or methylene and

R⁹, R¹⁰ and R¹¹ are each independently C₁-C₁₂-alkyl, C₅-C₁₅-arylalkyl or C₄-C₁₄-aryl and

R³ and R⁴ are each independently R², OR¹³ or NR¹⁴R¹⁵ where R², R¹³, R¹⁴ and R¹⁵ are each independently C₁-C₁₂-alkyl, C₅-C₁₅-arylalkyl or C₄-C₁₄-aryl, or NR¹⁴R¹⁵ together is a cyclic amino radical having 4 to 20 carbon atoms, or R³ and R⁴ together are —O—R¹⁶—O— where R¹⁶ is a radical selected from the group of C₂-C₄-alkylene, 1,2-phenylene, 1,3-phenylene, 1,2-cyclohexylene, 1,1′-ferrocenylene, 1,2-ferrocenylene, 2,2′-(1,1-binaphthylene), 2,2′-(1,1′)-biphenylene and 1,1′-(diphenyl-2,2′-methylene) diyl, and the radicals mentioned may optionally be mono- or polysubstituted by radicals selected from the group of fluorine, chlorine, C₁-C₈-alkoxy and C₁-C₈-alkyl and

R⁵ is hydrogen, C₁-C₂₀-alkyl, C₄-C₂₄-aryl, C₅-C₂₅-alylalkyl, C₁-C₂₀-haloalkyl or a radical of the formula (IIb)

A-B-D (IIb)

where

A is absent or is C₁-C₁₂-alkylene

B is a functionality which is selected from the group of

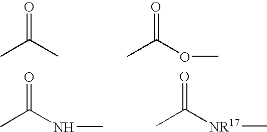

where

R¹⁷ may be C₁-C₂₀-alkyl, C₄-C₂₄-aryl, C₅-C₂₅-arylalkyl and

D is C₁-C₈-alkyl, C₄-C₂₄-aryl or C₅-C₂₅-arylalkyl or

B and D, in the case that A is not absent, may together be cyano or [(C₁-C₈-alkylene)-O]ₙ-(C₁-C₈-alkyl) where n is an integer between 1 and 8 or

R¹⁷ and D together are a cyclic amino radical having 4 to 12 carbon atoms.

For the purposes of the invention, all of the general or preferred radical definitions, parameters and illustrations above and cited hereinbelow, i.e. the particular areas and areas of preference also, may be combined as desired.

### DETAILED DESCRIPTION OF THE INVENTION

Alkyl, alkylene, alkoxy and alkenyl are each independently a straight-chain, cyclic, branched or unbranched alkyl, alkylene, alkoxy and alkenyl radical respectively, and each of the radicals mentioned may optionally also be substituted by C₁-C₄-alkoxy radicals.

The same applies to the nonaromatic moiety of an aralkyl radical.

C₁-C₄-alkyl is, for example, methyl, ethyl, 2-methoxyethyl, n-propyl, isopropyl, n-butyl, sec-butyl and tert-butyl, C₁-C₈-alkyl is additionally, for example, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, 1-ethyl-2-methylpropyl, n-heptyl and n-octyl, C₁-C₁₂-alkyl is further additionally, for example, adamantyl, the isomeric menthyls, n-nonyl, n-decyl and n-dodecyl, and C₁-C₂₀-alkyl is still further additionally, for example, n-hexadecyl and n-octadecyl.

C₁-C₈-alkoxy is, for example, methoxy, ethoxy, 2-methoxyethoxy, n-propoxy, isopropoxy, n-butoxy, sec-butoxy and tert-butoxy, n-pentoxy, 1-methylbutoxy, 2-methylbutoxy, 3-methylbutoxy, neopentoxy, 1-ethylpropoxy, cyclohexoxy, cyclopentoxy, n-hexoxy and n-octoxy, and C₁-C₁₂-alkoxy is further additionally, for example, adamantoxy, the isomeric menthoxy radicals, n-decoxy and n-dodecoxy.

C₂-C₂₀-alkenyl is, for example, vinyl, 1-propenyl, isopropenyl, 1-butenyl, 2-butenyl, 1-pentenyl, 2-pentenyl, 2-methyl-1-butenyl, 2-methyl-2-butenyl, 3-methyl-1-butenyl, 1-hexenyl, 1-heptenyl, 1-octenyl or 2-octenyl.

Haloalkyl is in each case independently a straight-chain, cyclic, branched or unbranched alkyl radical which is singly, multiply, or fully substituted by chlorine or fluorine atoms.

C₁-C₂₀-haloalkyl is, for example, trifluoromethyl, 2,2,2-trichloroethyl, 2,2,2-trifluoroethyl, pentafluoroethyl, nonafluorobutyl, perfluorooctyl, perfluorododecyl and perfluorohexadecyl.

Aryl is in each case independently a heteroaromatic radical having 5 to 18 framework carbon atoms of which no, one, two or three framework atoms per cycle, but at least one framework carbon atom in the entire molecule, may be substituted by heteroatoms selected from the group of nitrogen, sulphur or oxygen, but is preferably a carbocyclic aromatic radical having 6 to 18 framework carbon atoms.

Examples of carbocyclic aromatic radicals having 6 to 18 framework carbon atoms are phenyl, naphthyl, phenanthrenyl, anthracenyl or fluorenyl, and heteroaromatic radicals having 5 to 18 framework carbon atoms on which no, one, two or three framework carbon atoms per cycle, but at least one framework carbon atom in the entire molecule, may be substituted by heteroatoms selected from the group of nitrogen, sulphur or oxygen are, for example, pyridinyl, oxazolyl, benzofuranyl, dibenzofuranyl or quinolinyl.

The carbocyclic aromatic radical or heteroaromatic radical may also be substituted by up to five identical or different substituents per cycle which are selected from the group of chlorine, fluorine, C₁-C₁₂-alkyl, C₁-C₁₂-haloalkyl, C₁-C₁₂-alkoxy, di(C₁-C₈-alkyl)amino, COO(C₁-C₈-alkyl), CON(C₁-C₈-alkyl)₂, COO(C₁-C₈-arylalkyl), COO(C₄-C₁₄-aryl), CO(C₁-C₈-alkyl), C₅-C₁₅-arylalkyl or tri(C₁-C₆-alkyl)siloxyl.

The same applies to Aryloxy radicals.

Arylalkyl is in each case independently a straight-chain, cyclic or branched or unbranched alkyl radical which may be singly, multiply or fully substituted by aryl radicals as defined above.

C₅-C₂₅—Arylalkyl is, for example, benzyl, diphenylbenzyl, triphenylbenzyl (trityl), 1-phenylethyl, 1-phenylpropyl, 2-phenylpropyl, 1-phenyl-1-methylethyl, 1-, 2-, 3- or 4-phenylbutyl, 1-phenyl-1-methylpropyl, 1-phenyl-2-methylpropyl, phenyl-1,1-dimethylethyl, 1-, 2-, 3-, 4- or 5-phenylpentyl, phenyl-1-methylbutyl, phenyl-2-methylbutyl, phenyl-3-methylbutyl, phenyl-2,2-dimethylpropyl, phenyl-1-ethylpropyl, 1-naphthylmethyl, 1-naphthylethyl, naphthyl-1-methylethyl, naphthylbutyl, naphthyl-1-methylpropyl, naphthyl-2-methylpropyl, naphthyl-1,1-dimethylethyl, naphthylpentyl, naphthyl-1-methylbutyl, naphthyl-2-methylbutyl, naphthyl-3-methylbutyl, naphthyl-2,2-dimethylpropyl or naphthyl-1-ethylpropyl, and also their isomeric or stereoisomeric forms.

Arylalkenyl is in each case independently a straight-chain, cyclic, branched or unbranched alkenyl radical which may be singly, multiply or fully substituted by aryl radicals as defined above.

C₆-C₂₆-Arylalkenyl is, for example, 1-phenylvinyl or 2-phenylvinyl.

The preferred substitution patterns for compounds of the formula (I) are defined hereinbelow:

*1, *2, *3, *4 together define the following stereoisomers of the central substituted furan ring:

(1R,2R,3R,4R), (1R,2R,3R,4S), (1R,2R,3S,4S), (1R,2S,3S,4S), (1R,2S,3R,4S), (1R,2S,3S,4R), (1R,2R,3S,4R), (1S,2S,3R,4S), (1S,2S,3S,4S), (1S,2S,3S,4R), (1S,2S,3R,4R), (1S,2R,3R,4R), (1S,2R,3S,4R), (1S,2R,3R,4S), (1S,2S,3R,4S), (1R,2R,3S,4R), preferably (1R,2R,3R,4R), (1R,2R,3R,4S), (1R,2S,3S,4S), (1R,2S,3S,4R), (1R,2R,3S,4R), (1S,2S,3R,4S), (1S,2S,3S,4S), (1S,2S,3S,4R), (1S,2R,3R,4R), (1S,2R,3R,4S), (1S,2S,3R,4S), (1R,2R,3S,4R).

R¹ and R² are preferably each independently hydrogen, C₁-C₄-alkyl, C₄-C₁₄-aryl, O—R⁷, O₂C—R⁷, where R⁷ is preferably C₁-C₁₂-alkyl, C₅-C₂₅-arylalkyl or C₄-C₁₄-aryl, or OSiR⁹R¹⁰R¹¹R, where R⁹, R¹⁰, and R¹¹ are preferably each independently C₁-C₁₂-alkyl or C₄-C₁₄-aryl.

R¹ and R² are particularly preferably each independently hydrogen, tert-butoxy, trityloxy, tert-butyldimethylsilyloxy, tert-butyldiphenylsilyloxy, trimethylsilyloxy, triethylsilyloxy, triisopropylsilyloxy, neopentoxy or 1-adamantoxy.

For the purposes of the invention, preference is in each case given to those compounds of the formula (I) in which R¹ and R² are identical.

R³ and R⁴ are preferably each independently R¹², OR¹³ or NR¹⁴R¹⁵ where R¹², R¹³, R¹⁴ and R¹⁵ are each independently C₁-C₁₂-alkyl or C₄-C₁₄-aryl, or NR¹⁴R¹⁵ together is a cyclic amino radical having 4 to 12 carbon atoms, for example pyrrolidinyl or piperidinyl or R³ and R⁴ together are —O—R¹⁶—O— where R¹⁶ is ethylene, 1,2-phenylene, 1,3-phenylene, 1,2-cyclohexylene, 1,1′-ferrocenylene, di- or tetra-C₁-C₈-alkyl-substituted 1,1′-(diphenyl-2,2′-methylene)diyl, 1,2-ferrocenylene, 2,2′-(1,1′-binaphthylene) or 2,2′-(1,1′)-biphenylene, and 2,2′-(1,1′-binaphthylene) or 2,2′-(1,1′)-biphenylene is substituted at least in the 6,6′-position by radicals which are selected from the group of C₁-C₈-alkoxy and C₁-C₈-alkyl, and may also be substituted in the 5,5′-, 4,4′-, 3,3′- or 2,2′-position by radicals which are selected from the group of fluorine, chlorine, C₁-C₈-alkoxy and C₁-C₈-alkyl.

R³ and R⁴ are particularly preferably each independently R¹², OR¹³ or NR¹⁴R¹⁵, where R¹² and R¹³ are each independently methyl, ethyl, n-propyl, isopropyl, tert-butyl, cyclohexyl, phenyl, 2-(C₁-C₈)-alkylphenyl such as o-tolyl, 3-(C₁-C₈)-alkylphenyl such as m-tolyl, 4-(C₁-C₈)-alkylphenyl such as p-tolyl, 2,6-di-(C₁-C₈)-alkylphenyl such as 2,6-dimethylphenyl, 2,4-di-(C₁-C₈)-alkylphenyl such as 2,4-dimethylphenyl, 3,5-di-(C₁-C₈)-alkylphenyl such as 3,5-dimethylphenyl, 3,4,5-tri-(C₁-C₈)-alkylphenyl such as mesityl and isityl, 2-(C₁-C₈)-alkoxyphenyl such as o-anisyl and o-phenetyl, 3-(C₁-C₈)-alkoxyphenyl such as m-anisyl and m-phenetyl, 4-(C₁-C₈)-alkoxyphenyl such as p-anisyl and p-phenetyl, 2,4-di-(C₁-C₈)-alkoxyphenyl such as 2,4-dimethoxyphenyl, 2,6-Di-(C₁-C₈)-alkoxyphenyl such as 2,6-dimethoxyphenyl, 3,5-di-(C₁-C₈)-alkoxyphenyl such as 3,5-dimethoxyphenyl, 3,4,5-tri-(C₁-C₈)-alkoxyphenyl such as 3,4,5-trimethoxyphenyl, 3,5-dialkyl-4-(C₁-C₈)-alkoxyphenyl such as 3,5-dimethyl-4-anisyl, 3,5-(C₁-C₈)-dialkyl-4-di-(C₁-C₈)-alkylaminophenyl, 3,5-dimethyl-4-dimethylaminophenyl, 4-di-(C₁-C₈)-alkylaminophenyl such as 4-diethylaminophenyl and 4-dimethylaminophenyl, 3,5-bis-[(C₁-C₄)-fluoroalkyl]phenyl such as 3,5-bis-trifluoromethylphenyl, 2,4-bis-[(C₁-C₄)-fluoroalkyl]phenyl such as 2,4-bis-trifluoromethylphenyl, 4-[(C₁-C₄)-fluoroalkyl]phenyl such as 4-trifluoromethylphenyl and mono-, di-, tri-, tetra- or pentafluorine- and/or -chlorine-substituted phenyl, fluorenyl or naphthyl, such as 4-fluorophenyl and 4-chlorophenyl, and NR¹⁴R¹⁵ as a whole is dimethylamino, diethylamino, pyrrolidino or diisopropylamino. Particular preference is further given to R³ and R⁴ as a pair being O—R¹⁶—O, where R¹⁶ is 1,1′-bis-(4,6-di-(C₁-C₈-alkyl)phenyl)-2,2′-methylene)diyl, in particular 1,1′-bis-(4-methyl-6-tert-butylphenyl-2,2′-methylene)diyl and 1,1′-bis-(4-methyl-6-(1-methylcyclohexyl)phenyl-2,2′-methylene)diyl, or where R¹⁶ is (R)-1,1′-biphenyl-2,2′-diyl, (S)-1,1′-biphenyl-2,2′-diyl, (R)-1,1′-binaphthyl-2,2′-diyl, (S)-1,1′-binaphthyl-2,2′-diyl, 1,1′-[bis-(4-methyl-6-tert-butylphenyl)-2,2′-methylene)]diyl or 1,1′-[bis-(4-methyl-6-(1-methylcyclohexyl)-2,2′-methylene)]diyl.

R³ and R⁴ are very particularly preferably identical and are each 2,4-dimethylphenyl.

R⁵ is preferably hydrogen, C₁-C₄-alkyl, —CO(C₁-C₄-alkyl), benzyl-CO-phenyl or phenyl, and benzyl or phenyl may optionally be further substituted by one, two or three substituents selected from the group of C₁-C₄-alkyl, C₁-C₄-alkoxy or C₁-C₄-haloalkyl.

R⁵ is particularly preferably hydrogen, methyl or ethyl.

Particularly preferred compounds of the formula (I) are those of the formulae (Ia) to (Id)

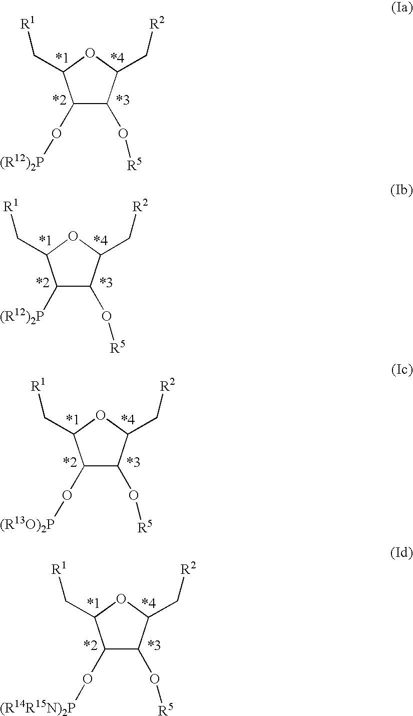

where *1, *2, *3, *4, R¹, R², R⁵, R¹², R¹³, R¹⁴ and R¹⁵ are as defined under formula (I).

A particularly preferred compound of the formula (I) is 2-O-(di(2,4-dimethylphenyl)phosphino)-1,6-di-O-(tert-butyldiphenylsilyl)-2,5-anhydro-D-mannitol.

For the purposes of the invention, the term stereoisomerically enriched includes stereoisomerically pure compounds or else mixtures of stereoisomeric compounds in which one stereoisomer is present in a greater relative proportion than the other stereoisomer(s), preferably in a relative proportion of 50 to 100 mol %, more preferably 90 to 100 mol % and most preferably 98 to 100 mol %, and includes in particular enantiomerically enriched compounds for which the same definitions apply.

The compounds of the formula (I) or (Ia) to (Id) can be prepared starting from the known 2,5-anhydrocyclopentoses of the formula (III).

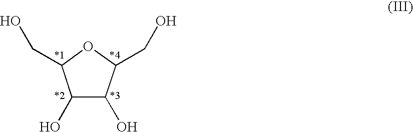

2,5-Anhydrocyclopentoses of the formula (III) are, for example:

2,5-anhydro-D-mannitol, 2,5-anhydro-L-mannitol, 2,5-anhydro-L-iditol, 2,5-anhydro-D-iditol, 2,5-anhydro-L-glucitol, 2,5-anhydro-D-glucitol, 2,5-anhydroaltritol, 2,5-anhydro-D-altritol, 2,5-anhydrogalactitol, 2,5-anhydroallitol.

Preferred compounds of the general formula (III) are:

2,5-anhydro-D-mannitol and 2,5-anhydro-L-iditol.

For the purposes of the invention, preference is given in particular to those compounds of the formula (I) which are obtainable starting from 2,5-anhydro-D-mannitol and 2,5-anhydro-L-iditol by the methods described hereinbelow.

The compounds of the formula (III) can be converted by reacting with compounds of the formula (IV)

R¹⁸-Hal (IV)

where R¹⁸ is R⁷, R⁷CO or OSiR⁹R¹⁰OR¹¹ and where R⁷, R⁹, R¹⁰ and R¹¹ each have the definition and areas of preference specified under formula (I) or R¹⁸ is R¹⁹—SO₂— where

R¹⁹ is C₁-C₁₂-alkyl, C₁-C₁₂-fluoroalkyl, C₅-C₂₅-arylalkyl or C₄-C₂₄-aryl and

Hal is chlorine, bromine or iodine

to compounds of the formula (V)

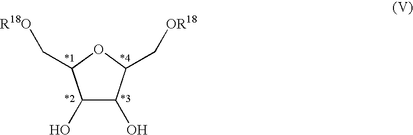

where

R¹⁸ is in each case independently as defined under formula (IV).

Compounds of the formula (V) in which R¹⁸ is R¹⁹SO₂— can also be converted by reacting with amines of the formula (VI)

HNR¹R⁷ (VI)

where

R⁷ and R⁸ each independently have the definitions and areas of preference specified under formula (I) to compounds of the formula (VII)

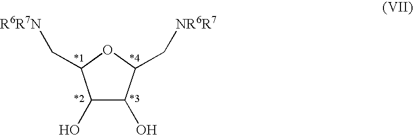

where

R⁶ and R⁷ are each independently as defined under formula (IV).

Compounds of formula (V) in which R¹⁸ is R¹⁹SO₂— can also be converted by reacting with complex hydrides of the formula (VM)

Met¹(AlR²⁰ₙR²¹₍₄₋ₙ₎) (VIII)

where

Met¹ is lithium, sodium or potassium, preferably lithium,

R²⁰ is hydrogen

n is 1, 2, 3 or 4, preferably 4 and

R²¹ is C₁-C₄-alkyl

or by reacting with organolithium compounds of the formula (IX)

R²⁰-Li (IX)

where

R²⁰ is C₁-C₂₀-alkyl, C₁-C₂₀-fluoroalkyl, C₂-C₂₀-alkenyl, C₄-C₂₄-aryl, C₅-C₂₅-arylalkyl, C₆-C₂₆-arylalkenyl, —(C₁-C₈-alkyl)-OR⁸, —(C₁-C₈-alkyl)-NR⁷R⁸ or protected (for example as a cyclic acetal)-(C₁-C₈-alkyl)-CO—R⁸ to compounds of the formula (X)

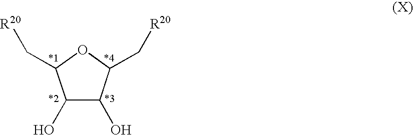

where

R²⁰ is as defined under formulae (VIII) and (IX).

As a consequence of the acidity of the free 2- and 3-hydroxyl groups, it is advantageous to use an excess of the organolithium compounds or of the complex hydrides, or to protect the 3,4-diol unit in a manner known per se by conversion, for example, to a cyclic acetal and subsequent deprotection.

The compounds of the formulae (V), (VII) and (X) together are encompassed by the compounds of the formula (XI) which can be used as intermediates in preparing the compounds of the formula (I) according to the invention.

In formula (XI)

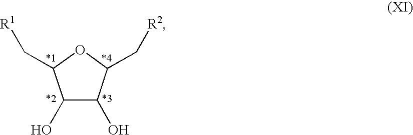

R¹ and R² have the same definition and areas of preference as described under formula (I).

The compounds of the formula (XI) can be used in a manner known in principle (see also Rajan Babu, J. Org. Chem., 1997, 62, 6012-6028) by reacting with compounds of the formula (XII)

R³R⁴P—Y (XII)

where

R³ and R⁴ have the same definition and areas of preference as specified under formula (I) and

Y is chlorine, bromine, iodine, dimethylamino or diethylamino, preferably chlorine,

to obtain the compounds of the formula (Ie)

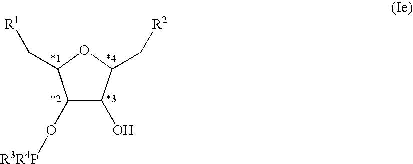

where

R¹, R², R³ and R⁴ each have the same definition and areas of preference as described under formula (I).

The compounds of the formula (Ie) can also be reacted with compounds of the formula (XIII)

R⁵Z (XIII)

where

R⁵ has the same definitions and areas of preference as specified under formula (I) and

Z is chlorine, bromine, iodine or R¹⁹SO₃ and, in the case that R⁵ is to be bonded via a carbonyl group, may also be R⁵O—

to give compounds of the formula (If),

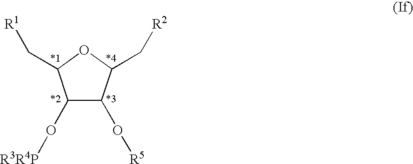

where

R¹, R², R³, R⁴, and R⁶ each have the same definitions and areas of preference as specified under formula (I) and R⁵ is not hydrogen.

Advantageously, the conversion to compounds of the formula (Ie) or (If) is effected after at least partial deprotonation of the alcohol function or in the presence of a base which can at least partially deprotonate the alcohol function.

Preferred bases for the conversion to compounds of the formula (Ie) are amines or N-heteroaromatics, in particular pyridine, and for the conversion to compounds of the formula (If), carbonates, hydroxides, alkoxides, amides and hydrides of alkali metals or alkaline earth metals, or amines or N-heteroaromatics, in particular pyridine.

Examples of suitable solvents for the conversion to compounds of the formula (Ie) are chlorinated alkanes such as methylene chloride, aliphatic hydrocarbons, e.g. hexane, cyclohexane, optionally chlorinated aromatic hydrocarbons, e.g. chlorobenzene, toluene, pyridine, benzene, ketones, e.g. acetone, or carboxylic esters, e.g. ethyl acetate, or dialkyl ethers, e.g. THF or methyl tert-butyl ether. The solvent used is preferably methylene chloride.

Suitable solvents for the conversion to compounds of the formula (If) are in principle the same solvents as for the conversion to compounds of the formula (Ie), although when strong bases such as hydroxides, alkoxides, amides and hydrides are used, it is advantageous to use no chlorinated alkanes.

The invention in particular also encompasses the compounds of the formula (If). The same definitions and areas of preference specified under formula (I) apply. However, in a preferred embodiment, the compounds of the formula (If) are prepared by initially reacting compounds of the formula (Ie) with compounds of the formula (XIII) to give compounds of the formula (XIV)

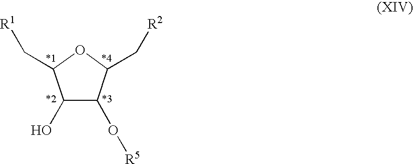

where

R¹, R², R³, R⁴, and R⁶ each have the same definitions and areas of preference as specified under formula (I) and R⁵ is not hydrogen, and then reacting the compounds of the formula (XIV) with compounds of the formula (XII) to give compounds of the formula (If).

The same information on solvents and bases applies as for the process via the compounds of the formula (Ie).

The compounds of the formula (XIV) are likewise encompassed by the invention.

Compounds of the formula (Ib)

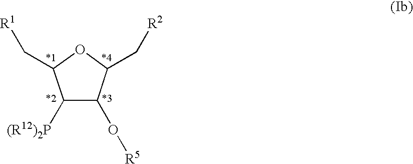

where

R¹, R², R⁵, R⁶ and R¹² each have the definitions and areas of preference specified under formula (I) can also be prepared by a process according to the invention by converting compounds of the formula (XV)

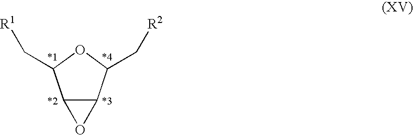

where

R¹ and R² have the definition and areas of preference specified under formula (I), in the presence of compounds of the formula (XVI),

(R¹²)₂PMet² (XVI)

where

Met² is lithium, sodium or potassium and

R¹² has the definition and areas of preference specified under formula (I)

to compounds of the formula (XVII),

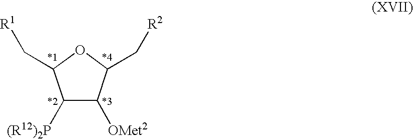

where

R¹, R², Met² and R¹² are each as defined above

and reacting the compounds of the formula (XVII) with compounds of the formula (XIII) as defined there to give compounds of the formula (Ib).

Alternatively, the compounds of the formula (XVII) can be converted by acidifying to compounds of the formula (Ib) in which R⁵ is hydrogen.

The compounds of the formula (Ib) can also be prepared, for example, by initially converting the compounds of the formula (XIV)

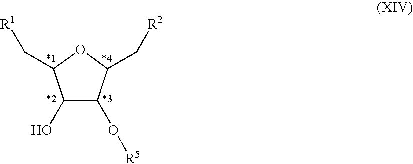

as defined above in a manner known per se (see also Terfort, Synthesis, 1992, 951-953) to compounds of the formula (XVIII)

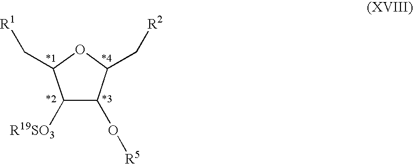

where

R¹ and R² each have the definition and areas of preference specified under formula (I) and R¹⁹ has the definition and areas of preference specified under formula (IV), and then reacting the compounds of the formula (XVIII) with phosphides of the formula (XVI).

The compounds of the formula (XVIII) are likewise encompassed by the invention.

The invention further encompasses transition metal complexes which contain the compounds of the formula (I) according to the invention.

Transition metal complexes are preferably those of ruthenium, osmium, cobalt, rhodium, iridium, nickel, palladium, platinum and copper, preferably those of ruthenium, rhodium, iridium, nickel, palladium, platinum and copper.

The transition metal complexes according to the invention are suitable in particular as catalysts. The invention therefore also encompasses catalysts which contain the transition metal complexes according to the invention.

The catalysts used may, for example, either be isolated transition metal complexes or those transition metal complexes which are obtainable by reacting transition metal compounds and compounds of the formula (I).

Isolated transition metal complexes which contain the compounds of the formula (I) are preferably those in which the ratio of transition metal to compound of the formula (I) is 1:2, 1:3 or 1:4.

Preference is given to the compounds according to the invention of the formula (XIX)

[(I)₄M] (XIX)

where

(I) is a compound of the formula (I) with the definition and the areas of preference specified there and

M is rhodium or iridium.

Preferred transition metal complexes are those which are obtainable by reacting transition metal compounds and compounds of the formula (I).

Suitable transition metal compounds are, for example, those of the formula (XXa)

M(An¹)_{q} (XXa)

where

M is rhodium, iridium, ruthenium, nickel, palladium, platinum or copper and

An¹ is chloride, bromide, acetate, nitrate, methanesulphonate, trifluoromethanesulphonate or acetylacetonate and

q is 3 for rhodium, iridium and ruthenium, is 2 for nickel, palladium and platinum, and is 1 for copper,

or transition metal compounds of the formula (XXb)

M(An²)_{q}L¹₂ (XXb)

where

M is ruthenium, iridium, ruthenium, nickel, palladium, platinum or copper and

An² is chloride, bromide, acetate, methanesulphonate or trifluoromethanesulphonate, tetrafluoroborate or hexafluorophosphate, perchlorate, hexafluoroantimonate, tetra(bis-3,5-trifluoromethylphenyl)-borate or tetraphenylborate and

q is 1 for rhodium and iridium, is 2 for ruthenium, nickel, palladium and platinum, and is 1 for copper,

L¹ is in each case C₂-C₁₂-alkene, for example ethylene or cyclooctene, or a nitrile, for example acetonitrile, benzonitrile or benzyl nitrile, or

L¹₂ together is a (C₄-C₁₂)-diene, for example bicyclo[2.1.1]hepta-2,5-diene (norbornadiene) or 1,5-cyclooctadiene,

or transition metal compounds of the formula (XXc)

[ML²An¹₂]₂ (XXc)

where

M is ruthenium and

L² is an aryl radical, for example cymene, mesityl, phenyl or cyclooctadiene, norbornadiene or methylallyl,

or transition metal compounds of the formula (XXd)

Met³_{q}[M(An³)₄] (XXd)

where

M is palladium, nickel, iridium or rhodium and

An³ is chloride or bromide and

Met³ is lithium, sodium, potassium, ammonium or an organic ammonium ion and

q is 3 for rhodium and iridium, and is 2 for nickel, palladium and platinum,

or transition metal compounds of the formula (XXe)

[M(L³)₂]An⁴ (XXe)

where

M is iridium or rhodium and

L³ is (C₄-C₁₂)-diene, for example bicyclo[2.1.1]hepta-2,5-diene (norbornadiene) or 1,5-cyclooctadiene and

An⁴ is a noncoordinating or weakly coordinating anion, for example methanesulphonate, trifluoromethanesulphonate, tetrafluoroborate, hexafluorophosphate, perchlorate, hexafluoroantimonate, tetra(bis-3,5-trifluoromethylphenyl)borate or tetraphenylborate.

Suitable transition metal compounds are additionally, for example, Ni(1,5-cyclooctadiene)₂, Pd₂(dibenzylideneacetone)₃, Pd[PPh₃]₄, cyclopentadienyl₂Ru, Rh(acac)(CO)₂, Ir(pyridine)₂(1,5-cyclooctadiene), Cu(phenyl)Br, Cu(phenyl)Cl, Cu(phenyl)I, Cu(PPh₃)₂Br, [Cu(CH₃CN)₄]BF₄ and [Cu(CH₃CN)₄]PF₆ or multinuclear bridged complexes, for example [Rh(1,5-cyclooctadiene)Cl]₂, [Rh(1,5-cyclooctadiene)Br]2, [Rh(ethene)₂Cl]₂, and [Rh(cyclooctene)₂C]₂.

The transition metal compounds used are preferably:

[Rh(cod)Cl]₂, [Rh(cod)Br]₂, [Rh(cod)₂]ClO₄, [Rh(cod)₂]BF₄, [Rh(cod)₂]PF₄, [Rh(cod)₂]ClO₆, [Rh(cod)₂]OTf, [Rh(cod)₂]BAr₄ (Ar=3,5-bistrifluoromethylphenyl), [Rh(cod)₂]SbF₆, RuCl₂(cod), [(cymene)RuCl₂]₂, [(benzene)RuCl₂]₂, [(mesityl)RuCl₂]₂, [(cymene)RuBr₂]₂, [(cymene)RuI₂]₂, [(cymene)Ru(BF₄)₂]₂, [(cymene)Ru(PF₆)₂]₂, [(cymene)Ru(BAr₄)₂]₂ (Ar=3,5-bistrifluoromethylphenyl), [(cymene)Ru(SbF₆)₂]₂, [Ir(cod)Cl]₂, [Ir(cod)₂]PF₆, [Ir(cod)₂]ClO₄, [Ir(cod)₂]SbF₆, [Ir(cod)₂]BF₄, [Ir(cod)₂]OTf, [Ir(cod)₂]BAr₄ (Ar=3,5-bistrifluoromethylphenyl), RuCl₃, NiCl₃, RhCl₃, PdCl₂, PdBr₂, Pd(OAc)₂, Pd₂(dibenzylideneacetone)₃, Pd(acetylacetonate)₂, CuOTf, CuI, CuCl, Cu(OTf)₂, CuBr, CuI, CuBr₂, CuCl₂, CuI₂, [Rh(nbd)Cl]₂, [Rh(nbd)Br]₂, [Rh(nbd)₂]ClO₄, [Rh(nbd)₂]BF₄, [Rh(nbd)₂]PF₆, [Rh(nbd)₂]OTf, [Rh(nbd)₂]BAr₄ (Ar=3,5-bistrifluoromethylphenyl), [Rh(nbd)₂]SbF₆, RuCl₂(nbd), [Ir(nbd)₂]PF₆, [Ir(nbd)₂]ClO₄, [Ir(nbd)₂]SbF₆, [Ir(nbd)₂]BF₄, [Ir(nbd)₂]OTf, [Ir(nbd)₂]BAr₄ (Ar=3,5-bistrifluoromethylphenyl), Ir(pyridine)₂(nbd), [Ru(DMSO)₄Cl₂], [Ru(CH₃CN)₄Cl₂], [Ru(PhCN)₄Cl₂], [Ru(cod)Cl₂]ₙ, [Ru(cod)₄(methallyl)₂], [Ru(acetylacetonate)₃].

Greater preference is given to [Rh(cod)Cl]₂, [Rh(cod)Br]₂, [Rh(cod)₂]ClO₄, [Rh(cod)₂]BF₄, [Rh(cod)₂]PF₄, [Rh(cod)₂]ClO₆, [Rh(cod)21OTf, [Rh(cod)₂]BAr₄ (Ar=3,5-bistrifluoromethylphenyl), [Rh(cod)₂]SbF₆, [Rh(nbd)Cl]₂, [Rh(nbd)Br]₂, [Rh(nbd)₂]ClO₄, [Rh(nbd)₂]BF₄, [Rh(nbd)₂]PF₆, [Rh(nbd)₂]OTf, [Rh(nbd)₂]BAr₄ (Ar=3,5-bistrifluoromethylphenyl), [Rh(nbd)₂]SbF₆, [Ir(cod)Cl]₂, [Ir(cod)₂]PF₆, [Ir(cod)₂]ClO₄, [Ir(cod)₂]SbF₆, [Ir(cod)₂]BF₄, [Ir(cod)₂]OTf, [Ir(cod)₂]BAr₄ (Ar=3,5-bistrifluoromethylphenyl).

The amount of the metal in the transition metal compounds used may, for example, be 5 to 100 mol %, based on the compound of the formula (1) used, preferably 10 to 50 mol % and most preferably 15 to 50 mol %.

The catalysts which contain the transition metal complexes according to the invention are suitable in particular for use in a process for preparing stereoisomerically enriched, preferably enantiomerically enriched, compounds.

Preference is given to using the catalysts for asymmetric 1,4-additions, asymmetric hydroformylations, asymmetric hydrocyanations, asymmetric Heck reactions and asymmetric hydrogenations, particular preference to using them for asymmetric hydrogenations.

Preferred asymmetric hydrogenations are, for example, hydrogenations of prochiral C═C-bonds, for example prochiral enamines, olefins, enol ethers, C═O bonds, for example prochiral ketones, and C═N bonds, for example prochiral imines. Particularly preferred asymmetric hydrogenations are hydrogenations of prochiral C═C bonds, for example prochiral enamines, olefins and C═N bonds, for example prochiral imines.

The invention therefore also encompasses a process for preparing stereoisomerically enriched, preferably enantiomerically enriched, compounds by catalytic hydrogenations of olefins, enamines, enamides, imines or ketones, which is characterized in that the catalysts used are those which contain transition metal complexes of compounds of the formula (I) as defined there.

The amount of the transition metal compound or of the transition metal complex used may, for example, be 0.001 to 5 mol %, based on the substrate used, preferably 0.001 to 0.5 mol %, very particularly preferably 0.001 to 0.1 mol % and even more preferably 0.001 to 0.008 mol %.

In a preferred embodiment, asymmetric hydrogenations can be carried out, for example, in such a way that the catalyst is formed from a transition metal compound and compound of the formula (I), optionally in a suitable solvent, the substrate is added and the reaction mixture is put under hydrogen pressure at room temperature.

The metal compounds used for asymmetric hydrogenations are particularly preferably those of general formula (XXI)

[M(L³)₂]An⁴ (XXI)

where M is rhodium or iridium and L³ and An are each as defined above,

or dinuclear complexes, for example [Rh(1,5-cyclooctadiene)Cl]₂, [Rh(1,5-cyclooctadiene)Br]₂, [Rh(ethene)₂Cl]₂, [Rh(cyclooctene)₂Cl]₂.

Particularly preferred metal compounds for asymmetric hydrogenations are [Rh(cod)₂]OTf, [Rh(cod)₂]BF₄, [Rh(cod)₂]PF₆, [Rh(nbd)₂]PF₆, [Rh(nbd)₂]BF₄, and [Rh(norbornadiene)₂]OTf, [Ir(cod)₂]BF₄ and [Ir(cod)₂ PF₆].

In a particularly preferred embodiment, transition metal compound and compound of the formula (I) are dissolved in degassed solvent in a baked-out glass autoclave. The mixture is stirred for approx. 5 min and the substrate is subsequently added in degassed solvent. After setting a particular temperature, hydrogenation is effected at elevated H₂ pressure.

Useful solvents for asymmetric catalysis are, for example, chlorinated alkanes such as methyl chloride, short-chain C₁-C₆-alcohols, e.g. methanol, isopropanol or ethanol, aromatic hydrocarbons, e.g. toluene or benzene, ketones, e.g. acetone, or carboxylic esters, e.g. ethyl acetate.

The asymmetric catalysis is advantageously carried out at a temperature of −20° C. to 200° C., preferably 0 to 100° C. and more preferably at 200 to 70° C.

The hydrogen pressure may, for example, be 0.1 to 200 bar, preferably 0.5 to 100 bar and more preferably 1 to 70 bar.

The catalysts according to the invention are suitable in particular in a process for preparing stereoisomerically enriched, preferably enantiomerically enriched, active ingredients in pharmaceuticals and agrochemicals, or intermediates of these two classes.

The advantage of the present invention is that ligands can be prepared in an efficient manner and their electronic and steric properties can be varied to a wide degree starting from readily available reactants. Furthermore, the ligands according to the invention and their transition metal complexes exhibit high enantioselectivities, especially in asymmetric hydrogenations of C═C bonds and imines.

### EXAMPLES

### Example 1

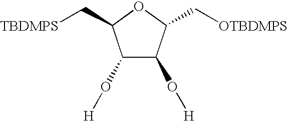

1,6-Di-O-(tert-butyldiphenylsilyl)-2,5-anhydro-D-mannitol (B1): 3 ml (11.66 mmol) of tert-butyldiphenylsilyl chloride (TBDMPSCl) were added dropwise at 0° C. to a solution of 0.87 g (5.3 mmol) of 2,5-anhydro-D-mannitol and 1.5 g (22.28 mmol) of imidazole in 12 ml of anhydrous DMF. The mixture was heated to room temperature and stirred for a further 25 hours, and the solvent was subsequently removed under reduced pressure. The mixture was diluted using CH₂Cl₂ and washed with water, and the organic phase was dried over Na₂SO₄ and the solvent subsequently removed under reduced pressure. The crude product was purified by means of column chromatography (4:1 hexane/ethyl acetate). Yield 1.36 g (40% of theory). ¹H NMR (400 MHz, CDCl₃) δ, 7.81-7.30 (m, 10H, Ph); 4.25 (m, 1H, H-3); 4.17 (m, 1H, H-2); 4.04 (d, 1H, OH); 3.86 (dd, 1H, J_{6,2}=3.7 Hz, J_{6,6}=11.1 Hz, H-6); 3.75 (dd, 1H, J_{6′,2}=3.2 Hz, J_{6,6′}=11.1 Hz, H-6′); 1.07 (s, 9H, C(CH₃)₃); ¹³C NMR (100.6 MHz) 8, 136.10-126.99 (Ph), 87.09 (C-2), 79.71 (C-3), 65.52 (C-6), 26.73 (C(CH₃)₃), 19.02 (C(CH₃)₃).

### Example 2

2-O-(Di(2,4-dimethylphenyl)phosphino)-1,6-di-O-(tert-butyldiphenylsilyl)-2,5-anhydro-D-mannitol (B2):

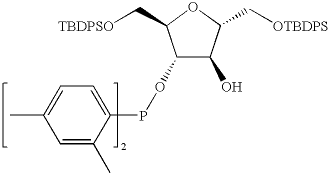

A solution of 169 mg (0.610 mmol) of bis-(2,4-dimethylphenyl)chlorophosphine in 2 ml of anhydrous THF was added to a solution of 300 mg (0.468 mmol) of 1,6-di-O-(tert-butyldiphenylsilyl)-2,5-anhydro-D-mannitol (B1) and 0.26 ml of anhydrous Et₃N (1.86 mmol) and stirred at room temperature overnight. After adding ethyl ether, the mixture was filtered through Celite®, the solvent removed under reduced pressure and the crude product purified by means of column chromatography. Yield 190 mg (49% of theory). ¹H NMR (400 MHz, CDCl₃) δ, 7.59-6.87 (m, 26H, arom.), 4.47 (m, 1H, CH), 4.31 (m, 1H, CH), 3.99 (m, 2H, CH), 3.69 (m, 3H, CH₂), 3.54 (dd, 1H, CH₂), 2.79 (s, OH), 2.30 (s, 3H, CH₃), 2.17 (s, 3H, CH₃), 2.11 (s, 3H, CH₃), 2.06 (s, 3H, CH₃), 0.96 (s, 9H, CH₃), 0.94 (s, 9H, CH₃). ¹³C NMR (75.4 MHz, CDCl₃) δ 138.1-127.6 (CH, C, arom.), 86.0 (²J_{C-P}=18 Hz, CH), 84.9 (CH), 83.9 (³J_{C-P}=6.13 Hz, CH), 78.0 (²J_{C-P}=4.5 Hz, CH CH), 64.7 (CH₂), 64.1 (CH₂), 27.1 (CH₃), 27.0 (CH₃), 21.4 (C), 20.5 (d, ³J=48.4 Hz, CH₃), 20.3 (d, ³J=48.4 Hz, CH₃), 19.6 (s, CH₃), 19.5 (s, CH₃). ³¹P NMR (161.9 MHz, CDCl₃) δ, 102.9.

Rhodium-Catalysed Hydrogenation of Enamides

### Examples 3 and 4

0.01 or 0.005 molar equivalent of transition metal compound and 0.027 molar equivalent of ligand were dissolved under argon in degassed CH₂Cl₂ (0.015 M) and stirred at room temperature for ½ hour. After adding one molar equivalent of substrate in degassed CH₂Cl₂ (0.08 M) under argon, the mixture obtained was hydrogenated in an autoclave at an appropriate temperature under hydrogen pressure. Conversion and ee were determined by chromatography.

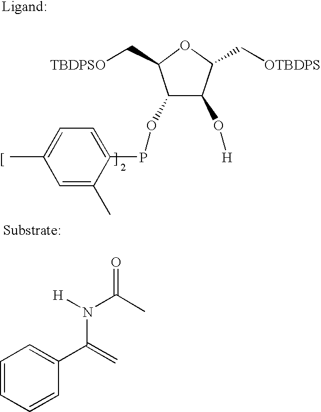

The results of the hydrogenations are compiled in Table 1.

TABLE 1

| | | | mol % | | | | Conver- | |
|---|---|---|---|---|---|---|---|---|
| Ex- | mol % of | | of metal | T | P | Time | sion | ee |
| ample | ligand | Metal precursor | precursor | (° C.) | (bar) | (h) | (%) | (%) |
| 3 | 2.7 | [Rh(nbd)₂]PF₆ | 1 | 25 | 3.5 | 16 | 96 | 18 |
| 4 | 2.7 | [Rh(cod)Cl₂]₂ | 0.5 | 25 | 30 | 24 | 100 | 83 |

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Compounds of the formula (I) 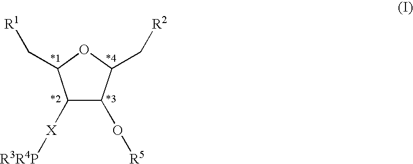
where
*1, *2, *3 and *4 are each independently a stereogenic carbon atom which has R- or S-configuration,
X is absent or is oxygen and
R¹and R² may each independently be hydrogen, C₁-C₂₀-alkyl, C₁-C₂₀-fluoroalkyl, C₂-C₂₀-alkenyl, C₄-C₂₄-aryl, C₅-C₂₅-arylalkyl, C₆-C₂₆-arylalkenyl or NR⁶R⁷, OR⁷, —(C₁-C₈-alkyl)-OR⁷, —(C₁-C₈-alkyl)-NR⁶R⁷ or —O₂CR⁷,
where R⁶ and R⁷ are each independently C₁-C₈-alkyl, C₅-C₁₅-arylalkyl or C₄-C₁₄-aryl, or R⁶ and R⁷ together are a cyclic amino radical having a total of 4 to 20 carbon atoms,
or R¹ and R² are each independently radicals of the formula (IIa)
—R⁸—SiR⁹R¹⁰R¹¹ (IIa)
where
R⁸ is absent or is oxygen or methylene and
R⁹, R¹⁰ and R¹¹ are each independently C₁-C₁₂-alkyl, C₅-C₁₅-arylalkyl or C₄-C₁₄-aryl and
R³ and R⁴ are each independently R¹², OR¹³ or NR¹⁴R¹⁵ where R¹², R¹³, R¹⁴ and R¹⁵ are each independently C₁-C₁₂-alkyl, C₅-C₁₅-arylalkyl or C₄-C₁₄-aryl, or NR¹⁴R¹⁵ together is a cyclic amino radical having 4 to 20 carbon atoms, or R³ and R⁴ together are —O—R¹⁶—O— where R¹⁶ is a radical selected from the group of C₂-C₄-alkylene, 1,2-phenylene, 1,3-phenylene, 1,2-cyclohexylene, 1,1′-ferrocenylene, 1,2-ferrocenylene, 2,2′-(1,1′-binaphthylene), 2,2′-(1,1′)-biphenylene and 1,1′-(diphenyl-2,2′-methylene)diyl, and the radicals mentioned may optionally be mono- or polysubstituted by radicals selected from the group of fluorine, chlorine, C₁-C₈-alkoxy and C₁-C₈-alkyl and
R⁵ is hydrogen, C₁-C₂₀-alkyl, C₄-C₂₄-aryl, C₅-C₂₅-arylalkyl, C₁-C₂₀-haloalkyl or a radical of the formula (IIb)
A-B-D (IIb)
where
A is absent or is C₁-C₁₂-alkylene
B is a functionality which is selected from the group of 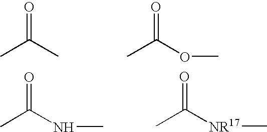
where
R¹⁷ may be C₁-C₂₀-alkyl, C₄-C₂₄-aryl, C₅-C₂₅-arylalkyl and
D is C₁-C₈-alkyl, C₄-C₂₄-aryl or C₅-C₂₅-arylalkyl or
B and D, in the case that A is not absent, are together optionally cyano or [(C₁-C₈-alkylene)-O]ₙ—(C₁-C₈-alkyl) where n is an integer between 1 and 8 or
R¹⁷ and D together are a cyclic amino radical having 4 to 12 carbon atoms.

2. Compounds according to claim 1, characterized in that *1, *2, *3 and *4 together define the following stereoisomers of the central substituted furan ring:
(1R,2R,3R,4R), (1R,2R,3R,4S), (1R,2S,3S,4S), (1R,2S,3S,4R), (1R,2R,3S,4R), (1S,2S,3R,4S), (1S,2S,3S,4S), (1S,2S,3S,4R), (1S,2R,3R,4R), (1S,2R,3R,4S), (1S,2S,3R,4S), (1R,2R,3S,4R).

3. Compounds according to claim 1, characterized in that R¹ and R² are each independently hydrogen, tert-butoxy, trityloxy, tert-butyldimethylsilyloxy, tert-butyldiphenylsilyloxy, trimethylsilyloxy, triethylsilyloxy, triisopropylsilyloxy, neopentoxy or 1-adamantoxy.

4. Compounds according to claim 1, characterized in that R¹ and R² are identical.

5. Compounds according to claim 1, characterized in that R³ and R⁴ are each independently R¹², OR¹³ or NR¹⁴R¹⁵ where R¹², R¹³, R¹⁴ and R¹⁵ are each independently C₁-C₁₂-alkyl or C₄-C₁₄-aryl, or NR¹⁴R¹⁵ together is a cyclic amino radical having 4 to 12 carbon atoms, or R³ and R⁴ together are —O—R¹⁶—O— where R¹⁶ is ethylene, 1,2-phenylene, 1,3-phenylene, 1,2-cyclohexylene, 1,1′-ferrocenylene, di- or tetra-C₁-C₈-alkyl-substituted 1,1′-(diphenyl-2,2′-methylene)diyl, 1,2-ferrocenylene, 2,2′-(1,1′-binaphthylene) or 2,2′-(1,1′)-biphenylene, and 2,2′-(1,1′-binaphthylene) or 2,2′-(1,1′)-biphenylene is substituted at least in the 6,6′-position by radicals which are selected from the group of C₁-C₈-alkoxy and C₁-C₈-alkyl, and is optionally substituted in the 5,5′-, 4,4′-, 3,3′- or 2,2′-position by radicals which are selected from the group of fluorine, chlorine, C₁-C₈-alkoxy and C₁-C₈-alkyl.

6. Compounds according to claim 1, characterized in that R⁵ is hydrogen, C₁-C₄-alkyl, —CO(C₁-C₄-alkyl), benzyl-CO-phenyl or phenyl, and benzyl or phenyl is optionally further substituted by one, two or three substituents selected from the group of C₁-C₄-alkyl, C₁-C₄-alkoxy or C₁-C₄-haloalkyl.

7. Compounds according to claim 1, characterized in that they are of the formulae (Ia) to (Id) 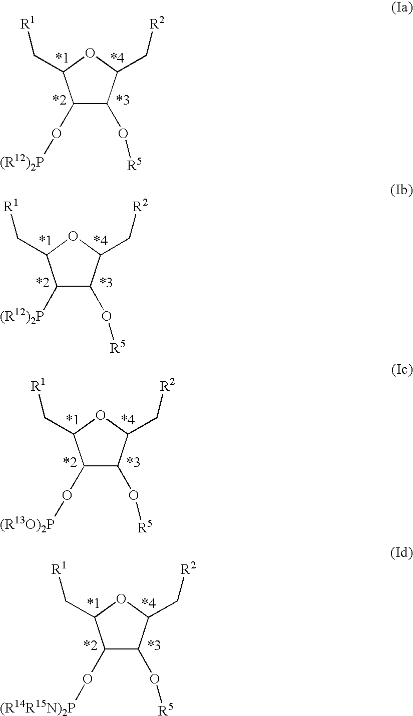
where *1, *2, *3, *4, R¹, R², R⁵, R¹², R¹³, R¹⁴ and R¹⁵ are as defined under formula (I).

8. 2-O-(Di(2,4-dimethylphenyl)phosphino)-1,6-di-O-(tert-butyldiphenylsilyl)-2,5-anhydro-D-mannitol.

9. Process for preparing compounds of the formula (Ib) 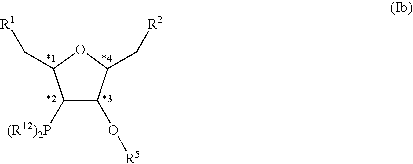
where
R¹, R², R⁵, R⁶ and R¹² are as defined under formula (I), comprising converting compounds of the formula (XV) 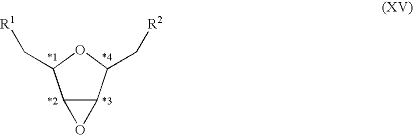
where
R¹ and R² are as defined under formula (I),
in the presence of compounds of the formula (XVI),
(R¹²)₂PMet² (XVI)
where
Met² is lithium, sodium or potassium and
R¹² has the definition specified under (I),
to compounds of the formula (XVII) 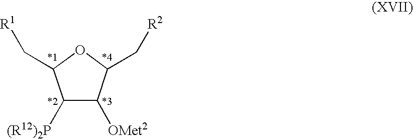
where
R¹, R², Met² and R¹² are as defined above, and
reacting the compounds of the formula (XVII) with compounds of the formula (XIII),
R⁵Z (XIII)
where
R⁵ has the same definitions as specified under formula (I) and
Z is chlorine, bromine, iodine or R¹⁹SO₃ where R¹⁹ is C₁-C₁₂-alkyl, C₁-C₁₂-haloalkyl, C₅-C₂₅-arylalkyl or C₄-C₂₄-aryl, and, in the case that R⁵ is to be bonded via a carbonyl group, is optionally R⁵O—.

10. Transition metal complexes containing compounds according to claim 1 and a transition metal compound.

11. Transition metal complexes according to claim 10, characterized in that the transition metal is selected from the group of ruthenium, osmium, cobalt, rhodium, iridium, nickel, palladium, platinum and copper.

12. Transition metal complexes according to claim 10, characterized in that the molar ratio of transition metal to the compounds is 1:2, 1:3 or 1:4.

13. Transition metal complexes according to claim 10, which is of the formula (XIX)
[(I)₄M] (XIX)
where
(I) is a compound of the formula (I) as defined in claim 1 and
M is rhodium or iridium.

14. Transition metal complexes according to claim 10 characterized in that they are obtained by reacting the transition metal compounds and the compounds.

15. Transition metal complexes according to claim 14, characterized in that the transition metal compounds used are:
transition metal compounds of the formula (XXa)
M(An¹)_{q} (XXa)
where
M is rhodium, iridium, ruthenium, nickel, palladium, platinum or copper and
An¹ is chloride, bromide, acetate, nitrate, methanesulphonate, trifluoromethanesulphonate or acetylacetonate and
q is 3 for rhodium, iridium and ruthenium, is 2 for nickel, palladium and platinum, and is I for copper,
or transition metal compounds of the formula (XXb)
M(An²)_{q}L¹₂ (XXb)
where
M is ruthenium, iridium, ruthenium, nickel, palladium, platinum or copper and
An² is chloride, bromide, acetate, methanesulphonate or trifluoromethanesulphonate, tetrafluoroborate or hexafluorophosphate, perchlorate, hexafluoroantimonate, tetra(bis-3,5-trifluoromethylphenyl)borate or tetraphenylborate and
q is 1 for rhodium and iridium, is 2 for ruthenium, nickel, palladium and platinum, and is 1 for copper,
L¹ is in each case C₂-C₁₂-alkene, , or a nitrile, or
L¹₂ together is a (C₄-C₁₂)-diene,,
or transition metal compounds of the formula (XXc)
[ML²An¹₂]₂ (XXc)
where
M is ruthenium and
L² is an aryl radical, or methylallyl,
or transition metal compounds of the formula (XXd)
Met³_{q}[M(An³)₄] (XXd)
where
M is palladium, nickel, iridium or rhodium and
An³ is chloride or bromide and
Met³ is lithium, sodium, potassium, ammonium or an organic ammonium ion and
q is 3 for rhodium and iridium, and is 2 for nickel, palladium and platinum,
or transition metal compounds of the formula (XXe)
[M(L³)₂]An⁴ (XXe)
where
M is iridium or rhodium and
L³ is (C₄-C]₂)-diene, and
An⁴ is an uncoordinating or weakly coordinating anion, methanesulphonate, trifluoromethanesulphonate, tetrafluoroborate, hexafluorophosphate, perchlorate, hexafluoroantimonate, tetra(bis-3,5-trifluoromethylphenyl)borate or tetraphenylborate,
or Ni(1,5-cyclooctadiene)₂, Pd₂(dibenzylideneacetone)₃, Pd[PPh₃]₄, cyclopentadienyl₂Ru, Rh(acac)(CO)₂, Ir(pyridine)2(1,5-cyclooctadiene), Cu(phenyl)Br, Cu(phenyl)Cl, Cu(phenyl)I, Cu(PPh₃)₂Br, [Cu(CH₃CN)₄]BF₄ and [Cu(CH₃CN)₄]PF₆ or multinuclear bridged complexes.

16. Transition metal complexes according to claim 14, characterized in that the amount of the metal in the transition metal compounds used is from 5 to 100 mol %.

17. Catalysts containing transition metal complexes according to claim 10.

18. A process for preparing stereoisomerically enriched compounds comprising providing transition metal complexes according to claim 10 or catalysts containing the transition metal complexes.

19. The process of o claim 18, characterized in that the stereoisomerically enriched compounds are obtained by asymmetric 1,4-additions, asymmetric hydroformylations, asymmetric hydrocyanations, asymmetric Heck reactions and asymmetric hydrogenations.

20. The process of claim 18, characterized in that the stereoisomerically enriched compounds are used for preparing active ingredients in pharmaceuticals and agrochemicals, or intermediates of both of these classes.

21. Process for preparing stereoisomerically enriched compounds by catalytic hydrogenations of olefins, enamines, enamides, imines or ketones, 1,4-additions, hydroformylations, hydrocyanations or Heck reactions, comprising providing catalysts which contain transition metal complexes according to claim 10.

22. Process according to claim 21, characterized in that the amount of the transition metal complexes used is 0.001 to 5 mol %, based on substrate used.

23. Process according to claim 21, characterized in that the stereoisomerically enriched compounds are obtained by catalytic hydrogenation of olefins, enamides or imines.

24. Process according to claim 21, characterized in that the working temperature is −20° C. to 200° C.

25. Process according to claim 21, characterized in that the hydrogen pressure is 0.1 to 200 bar.

26. Compounds of the formula (XIV) 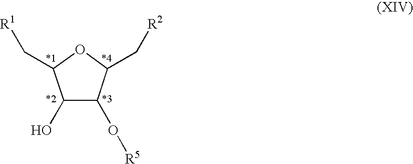
where
R¹, R² and R⁵ are each as defined under formula (I) in claim 1.

27. Compounds of the formula (XVIII) 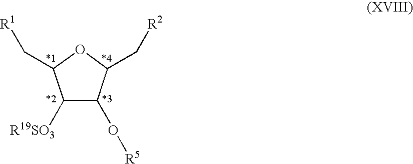
where
R¹, R² and R⁵ are each as defined under formula (I) in claim 1 and
R¹⁹ is C₁-C₁₂-alkyl, C₁-C₁₂-fluoroalkyl, C₅-C₂₅-arylalkyl or C₄-C₂₄-aryl.
